SEQUENCE LISTING (2 sequences)

SEQ ID NO: 1 - protein, 22 aa, Artificial
  A-chain
XGIVEXCCHSICSLYXLEXYCG

SEQ ID NO: 2 - protein, 34 aa, Artificial
  B-chain
XXXXXXHLCGSHLVEALYLVCGERGFFYTPXXXX

(19) United States
(12) Patent Application Publication
(10) Pub. No.: US 2012/0232002 A1
(43) Pub. Date: Sep. 13, 2012

(54) SLOW-ACTING INSULIN PREPARATIONS

(75) Inventors: Isabell Schoettle, Frankfurt am Main (DE); Christiane Fuerst, Frankfurt am Main (DE); Verena Siefke-Henzler, Frankfurt am Main (DE); Gerrit Hauck, Frankfurt am Main (DE); Walter Kamm, Frankfurt am Main (DE); Julia Schnieders, Frankfurt am Main (DE); Jutta Carls, Frankfurt am Main (DE); Gert Wolf, Frankfurt am Main (DE)

(73) Assignee: SANOFI-AVENTIS DEUTSCHLAND GMBH, Frankfurt (DE)

(21) Appl. No.: 13/382,772

(22) PCT Filed: Jul. 2, 2010
(86) PCT No.: PCT/EP10/59438

§ 371 Date: May 29, 2012

Related U.S. Application Data
(60) Provisional application No. 61/264,353, filed on Nov. 25, 2009.

(30) Foreign Application Priority Data
Jul. 6, 2009 (DE) 102009031750.3
Mar. 27, 2010 (DE) 102010013133.4

(51) Int. Cl. A61K 38/28; A61P 3/10

(52) U.S. Cl. 514/6.2

(57) ABSTRACT

Aqueous pharmaceutical formulations with an insulin analog, comprising
0.001 to 0.2 mg/ml of zinc,
0.1 to 5.0 mg/ml of a preservative, and
5.0 to 100 mg/ml of an isotonicity agent, and
   whose pH is 5 or less, and also their preparation, use for treating diabetes mellitus, and a medicament for treating diabetes mellitus.

The invention relates to an aqueous pharmaceutical formulations with an insulin analog comprising
0.001 to 0.2 mg/ml of zinc,
0.1 to 5.0 mg/ml of a preservative, and
5.0 to 100 mg/ml of an isotonicity agent, and
   whose pH is 5 or less; and also to its preparation, use for treating diabetes mellitus, and to a medicament for treating diabetes mellitus.

An increasing number of people around the world suffer from diabetes mellitus. Many of them are what are called type I diabetics, for whom replacement of the deficient endocrine insulin secretion is the only possible therapy at present. Those affected are dependent on insulin injections for life, usually several times a day. Type II diabetes contrasts with type I diabetes in that there is not always a deficiency of insulin, but in a large number of cases, especially at the advanced stage, treatment with insulin, where appropriate in combination with an oral antidiabetic, is considered the most advantageous form of therapy.

In healthy individuals, release of insulin by the pancreas is strictly coupled to the blood glucose concentration. Elevated blood glucose levels, like those occurring after meals, are quickly compensated by a corresponding rise in insulin secretion. In the fasting state, the plasma insulin level falls to a base line value which is sufficient to ensure a continuous supply of glucose to insulin-sensitive organs and tissues, and to keep hepatic glucose production low in the night. The replacement of the endogenous insulin secretion by exogenous, usually subcutaneous administration of insulin does not in general come close to the above-described quality of the physiological regulation of blood glucose. Frequently there are instances of blood glucose being thrown off-track, either upwardly or downwardly, and in their most severe forms these instances may be life-threatening. In addition, however, blood glucose levels which are elevated over years, without initial symptoms, constitute a considerable health risk. The large-scale DCCT study in the USA (The Diabetes Control and Complications Trial Research Group (1993), N. Engl. J. Med. 329, 977-986) showed unambiguously that chronically elevated blood glucose levels are responsible for the development of late diabetic complications. Late diabetic complications are microvascular and macrovascular damage which is manifested in certain circumstances as retinopathy, nephropathy, or neuropathy, and leads to blindness, renal failure, and loss of extremities, and, in addition, is associated with an increased risk of cardiovascular disorders. From this it can be inferred that an improved therapy of diabetes must be aimed primarily at keeping blood glucose as closely as possible within the physiological range. According to the concept of intensified insulin therapy, this is to be achieved by means of injections, several times a day, of fast-acting and slow-acting insulin preparations. Fast-acting formulations are given at meal times, in order to compensate the postprandial rise in blood glucose. Slow-acting basal insulins are intended to ensure the basic supply of insulin, especially during the night, without leading to hypoglycemia.

Insulin is a polypeptide composed of 51 amino acids which are divided between two amino acid chains: the A chain, with 21 amino acids, and the B chain, with 30 amino acids. The chains are linked together by two disulfide bridges. Insulin preparations have been employed for many years in diabetes therapy. Such preparations use not only naturally occurring insulins but also, more recently, insulin derivatives and insulin analogs.

Insulin analogs are analogs of naturally occurring insulins, namely human insulin or animal insulins, which differ by replacement of at least one naturally occurring amino acid residue by other amino acids and/or by addition/deletion of at least one amino acid residue, from the corresponding, otherwise identical, naturally occurring insulin. The amino acids in question may also be amino acids which do not occur naturally.

Insulin derivatives are derivatives of naturally occurring insulin or an insulin analog which are obtained by chemical modification. The chemical modification may consist, for example, in the addition of one or more defined chemical groups to one or more amino acids. Generally speaking, the activity of insulin derivatives and insulin analogs is somewhat altered as compared with human insulin.

Insulin analogs with an accelerated onset of action are described in EP 0 214 826, EP 0 375 437, and EP 0 678 522. EP 0 124 826 relates, among other things, to replacements of B27 and B28. EP 0 678 522 describes insulin analogs which have different amino acids in position B29, preferably proline, but not glutamic acid. EP 0 375 437 encompasses insulin analogs with lysine or arginine at B28, which may also optionally be modified at B3 and/or A21.

EP 0 419 504 discloses insulin analogs which are protected from chemical modifications by modification of asparagine in B3 and of at least one further amino acid at positions A5, A15, A18 or A21.

Generally speaking, insulin derivatives and insulin analogs have a somewhat altered action as compared with human insulin.

WO 92/00321 describes insulin analogs in which at least one amino acid in positions B1-B6 has been replaced by lysine or arginine. Such insulins, according to WO 92/00321, have an extended effect. A delayed effect is also exhibited by the insulin analogs described in EP-A 0 368 187. The concept of intensified insulin therapy attempts to reduce the risk to health by aiming for stable control of the blood sugar level by means of early administration of basal insulins. One example of a common basal insulin is the drug Lantus® (active ingredient: insulin glargine=Gly (A21), Arg (B31), Arg (B32) human insulin). Generally speaking, the aim in the development of new, improved basal insulins is to minimize the number of hypoglycemic events. An ideal basal insulin acts safely in each patient for at least 24 hours. Ideally, the onset of the insulin effect is delayed and has a fairly flat time/activity profile, thereby significantly minimizing the risk of short-term undersupply of sugar, and allowing administration even without food being taken beforehand. The supply of basal insulin is effective when the insulin activity goes on consistently for as long as possible, i.e., the body is supplied with a constant amount of insulin. As a result, the risk of hypoglycemic events is low, and patient-specific and day-specific variability are minimized. The pharmacookinetic profile of an ideal basal insulin, then, ought to be characterized by a delayed onset of action and by a delayed action, i.e., a long-lasting and uniform action.

The preparations of naturally occurring insulins for insulin replacement that are present on the market differ in the origin of the insulin (e.g., bovine, porcine, human insulin) and also in their composition, and so the activity profile (onset and duration of action) may be affected. Through combination of different insulin products it is possible to obtain any of a very wide variety of activity profiles and to bring about very largely physiological blood sugar values. Recombinant DNA technology nowadays allows the preparation of modified insulins of this kind. They include insulin glargine (Gly(A21)-Arg(B31)-Arg(B32) human insulin), with an extended duration of action. Insulin glargine is injected in the form of a clear, acidic solution, and, on the basis of its dissolution properties is precipitated, in the physiological pH range of the subcutaneous tissue, as a stable hexamer association. Insulin glargine is injected once a day and is notable in comparison with other long-active insulins for its flat serum profile and the associated reduction in the risk of night hypoglycemias (Schubert-Zsilavecz et al., 2:125-130 (2001)). In contrast to preparations described to date, the specific preparation of insulin glargine that leads to the prolonged duration of action is characterized by a clear solution with an acidic pH. Specifically at acidic pH, however, insulins exhibit reduced stability and an increased tendency toward aggregation under thermal and physico-mechanical load, which may be manifested in the form of haze and precipitation (particle formation) (Brange et al., J. Ph. Sci 86:517-525 (1997)).

It was an object of the present invention, therefore, to find further formulations for insulin analogs soluble in the acidic range, with a delayed onset of action and a prolonged duration of action, i.e., an activity profile which is extremely flat, long-lasting, and uniform. This further significantly minimizes the risk of hypoglycemic events.

It has surprisingly been found that such formulations lead to the described desired basal time/activity profile, when the insulin analogs are characterized by the features that
the B chain end is composed of an amidated basic amino acid residue such as lysine or arginine amide, i.e., in the amidated basic amino acid residue at the B chain end, the carboxyl group of the terminal amino acid is in its amidated form, and
the N-terminal amino acid residue of the insulin A chain is a lysine or arginine residue, and
the amino acid position A8 is occupied by a histidine residue, and
the amino acid position A21 is occupied by a glycine residue, and
there are two replacements of neutral amino acids by acidic amino acids, two additions of negatively charged amino acid residues, or one such replacement and one such addition, in each of positions A5, A15, A18, B-1, B0, B1, B2, B3, and B4; and comprise
0.001 to 0.2 mg/ml of zinc,
0.1 to 5.0 mg/ml of a preservative, and
5.0 to 100 mg/mi of an isotonicity agent, and
have a pH of 5 or less.

The invention accordingly provides an aqueous, pharmaceutical formulations having an insulin analog of the formula I

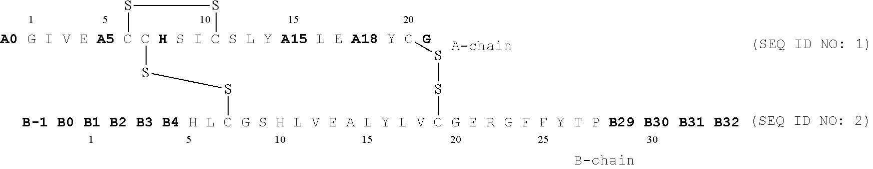

where
A0 is Lys or Arg;
A5 is Asp, Gln or Glu;
A15 is Asp, Glu or Gln;
A18 is Asp, Glu or Asn;
B-1 is Asp, Glu or an amino group;
B0 is Asp, Glu or a chemical bond;
B1 is Asp, Glu or Phe;
B2 is Asp, Glu or Val;
B3 is Asp, Glu or Asn;
B4 is Asp, Glu or Gln;
B29 is Lys or a chemical bond;
B30 is Thr or a chemical bond;
B31 is Arg, Lys or a chemical bond;
B32 is Arg-amide, Lys-amide or an amino group,
   where two amino acid residues of the group containing A5, A15, A18, B-1, B0, B1, B2, B3, and B4, simultaneously and independently of one another, are Asp or Glu, or a pharmacologically tolerable salt thereof; and which comprises
0.001 to 0.2 mg/ml of zinc,
0.1 to 5.0 mg/ml of a preservative, and
5.0 to 100 mg/ml of an isotonicity agent, and
   whose pH is 5 or less.

The invention further provides a pharmaceutical formulation as described above in which the insulin analog is selected from a group containing:
Arg (A0), His (A8), Glu (A5), Asp (A18), Gly (A21), Arg (B31), Arg (B32)-NH₂ human insulin,
Arg (A0), His (A8), Glu (A5), Asp (A18), Gly (A21), Arg (B31), Lys (B32)-NH₂ human insulin,
Arg (A0), His (A8), Glu (A15), Asp (A18), Gly (A21), Arg (B31), Arg (B32)-NH₂ human insulin,
Arg (A0), His (A8), Glu (A15), Asp (A18), Gly (A21), Arg (B31), Lys (B32)—NH₂ human insulin,
Arg (A0), His (A8), Glu(A5), Glu (A15), Gly (A21), Arg (B31), Arg (B32)—NH₂ human insulin,
Arg (A0), His (A8), Glu (A5), Glu (A15), Gly (A21), Arg (B31), Lys (B32)-NH₂ human insulin,
Arg (A0), His(A8), Glu (A5), Gly (A21), Asp (B3), Arg (B31), Arg (B32)—NH₂ human insulin,
Arg (A0), His(A8), Glu (A5), Gly (A21), Asp (B3), Arg (B31), Lys (B32)-NH₂ human insulin,
Arg (A0), His (A8), Glu (A15), Gly (A21), Asp (B3), Arg (B31), Arg (B32)-NH₂ human insulin,
Arg (A0), His (A8), Glu (A15), Gly (A21), Asp (B3), Arg (B31), Lys (B32)-NH₂ human insulin,
Arg (A0), His (A8), Asp (A18), Gly (A21), Asp (B3), Arg (B31), Arg (B32)-NH₂ human insulin,
Arg (A0), His (A8), Asp (A18), Gly (A21), Asp (B3), Arg (B31), Lys (B32)-NH₂ human insulin,
Arg (A0), His(A8), Gly (A21), Asp (B3), Glu (B4), Arg (B31), Arg (B32)-NH₂ human insulin,
Arg (A0), His (A8), Gly (A21), Asp (B3), Glu (B4), Arg (B31), Lys (B32)-NH₂ human insulin,
Arg (A0), His (A8), Glu (A5), Gly (A21), Glu (B4), Arg (B31), Arg (B32)-NH₂ human insulin,
Arg (A0), His (A8), Glu (A5), Gly (A21), Glu (B4), Arg (B31), Lys (B32)-NH₂ human insulin,
Arg (A0), His (A8), Glu (A15), Gly (A21), Glu (B4), Arg (B31), Arg (B32)-NH₂ human insulin,
Arg (A0), His (A8), Glu (A15), Gly (A21), Glu (B4), Arg (B31), Lys (B32)-NH₂ human insulin,
Arg (A0), His (A8), Asp (A18), Gly (A21), Glu (B4), Arg (B31), Arg (B32)-NH₂ human insulin,
Arg (A0), His (A8), Asp (A18), Gly (A21), Glu (B4), Arg (B31), Lys (B32)-NH₂ human insulin,
Arg (A0), His (A8), Glu (A5), Gly (A21), Glu (B0), Arg (B31), Arg (B32)-NH₂ human insulin,
Arg (A0), His (A8), Glu (A5), Gly (A21), Glu (B0), Arg (B31), Lys (B32)-NH₂ human insulin,
Arg (A0), His (A8), Glu (A15), Gly (A21), Glu (B0), Arg (B31), Arg (B32)-NH₂ human insulin,
Arg (A0), His (A8), Glu (A15), Gly (A21), Glu (B0), Arg (B31), Lys (B32)-NH₂ human insulin,
Arg (A0), His (A8), Asp (A18), Gly (A21), Glu (B0), Arg (B31), Arg (B32)-NH₂ human insulin,
Arg (A0), His (A8), Asp (A18), Gly (A21), Glu (B0), Arg (B31), Lys (B32)-NH₂ human insulin,
Arg (A0), His (A8), Glu (A5), Gly (A21), Asp (B1), Arg (B31), Arg (B32)-NH₂ human insulin,
Arg (A0), His (A8), Glu (A5), Gly (A21), Asp (B1), Arg (B31), Lys (B32)-NH₂ human insulin,
Arg (A0), His (A8), Glu (A15), Gly (A21), Asp (B1), Arg (B31), Arg(B32)-NH₂ human insulin,
Arg (A0), His (A8), Glu (A15), Gly (A21), Asp (B1), Arg (B31), Lys (B32)-NH₂ human insulin,
Arg (A0), His (A8), Asp (A18), Gly (A21), Asp (B1), Arg (B31), Arg (B32)-NH₂ human insulin,
Arg (A0), His (A8), Asp (A18), Gly (A21), Asp (B1), Arg (B31), Lys (B32)-NH₂ human insulin,
Arg (A0), His (A8), Gly (A21), Glu (B0), Asp (B1), Arg (B31), Arg (B32)-NH₂ human insulin,
Arg (A0), His (A8), Gly (A21), Glu (B0), Asp (B1), Arg (B31), Lys (B32)-NH₂ human insulin,
Arg (A0), His (A8), Asp (A18), Gly (A21), Asp (B3), Arg (B30), Arg (B31)-NH₂human insulin,
Arg (A0), His (A8), Asp (A18), Gly (A21), Asp (B3), Arg (B30), Lys (B31)-NH₂ human insulin.

The invention further provides a pharmaceutical formulation as described above, the preservative being selected from a group containing phenol, m-cresol, chlorocresol, benzyl alcohol, and parabens.

The invention further provides a pharmaceutical formulation as described above, the isotonicity agent being selected from a group containing mannitol, sorbitol, lactose, dextrose, trehalose, sodium chloride, and glycerol.

The invention further provides a pharmaceutical formulation as described above, having a pH in the range of pH 2.5-4.5, preferably in the range of pH 3.0-4.0, more preferably in the region of pH 3.75.

The invention further provides a pharmaceutical formulation as described above, the insulin, insulin analog and/or insulin derivative being present in a concentration of 60-6000 nmol/ml.

The invention further provides a pharmaceutical formulation as described above, comprising glycerol at a concentration of 20 to 30 mg/ml, preferably at a concentration of 25 mg/ml.

The invention further provides a pharmaceutical formulation as described above, comprising m-cresol at a concentration of 1 to 3 mg/ml, preferably at a concentration of 2 mg/ml.

The invention further provides a pharmaceutical formulation as described above, comprising zinc at a concentration of 0.01 or 0.03 or 0.08 mg/ml.

The invention further provides a pharmaceutical formulation as described above, further comprising a glucagon-like peptide-1 (GLP1) or an analog or derivative thereof, or exendin-3 and/or -4 or an analog or derivative thereof.

The invention further provides a pharmaceutical formulation as described above, further comprising exendin-4.

The invention further provides a pharmaceutical formulation as described above, in which an analog of exendin-4 is selected from a group containing
H-desPro³⁶-exendin-4-Lys₆-N H₂,
H-des(Pro^{36,37})-exendin-4-Lys₄-N H₂ and
H-des(Pro^{36,37})-exendin-4-Lys₅-N H₂,
   or a pharmacologically tolerable salt thereof.

The invention further provides a pharmaceutical formulation as defined above in which an analog of exendin-4 is selected from the group containing
desPro³⁶[Asp²⁸]exendin-4 (1-39),
desPro³⁶[IsoAsp²⁸]exendin-4 (1-39),
desPro³⁶[Met(O)¹⁴, Asp²⁸]exendin-4 (1-39),
desPro³⁶[Met(O)¹⁴, IsoAsp²⁸]exendin-4 (1-39),
desPro³⁶[Trp(O₂)²⁵, Asp²⁸]exendin-2 (1-39),
desPro³⁶[Trp(O₂)²⁵, IsoAsp²⁸]exendin-2 (1-39),
desPro³⁶[Met(O)¹⁴Trp(O₂)²⁵, Asp²⁸]exendin-4 (1-39) and
desPro³⁶[Met(O)¹⁴Trp(O₂)²⁵, IsoAsp²⁸]exendin-4 (1-39),
   or a pharmacologically tolerable salt thereof.

The invention further provides a pharmaceutical formulation as described above in which the peptide Lys₆-NH₂ is attached to the C-termini of the analogs of exendin-4.

The invention further provides a pharmaceutical formulation as described above, in which an analog of exendin-4 is selected from the group containing
H-(Lys)₆-des Pro³⁶[Asp²⁸]exendin-4(1-39)-Lys₆-NH₂
des Asp²⁸Pro³⁶, Pro³⁷, Pro³⁸exendin-4(1-39)-NH₂,
H-(Lys)₆-des Pro³⁶, Pro³⁷, Pro³⁸[Asp²⁸]exendin-4(1-39)-NH₂,
H-Asn-(Glu)₅des Pro³⁶, Pro³⁷, Pro³⁸[Asp²⁸]exendin-4(1-39)-NH₂,
des Pro³⁶, Pro³⁷, Pro³⁸[Asp²⁸]exendin-4(1-39)-(Lys)₆-NH₂,
H-(Lys)₆-des Pro³⁶, Pro³⁷, Pro³⁸[Asp²⁸]exendin-4(1-39)-(Lys)₆-NH₂,
H-Asn-(Glu)₅-des Pro³⁶, Pro³⁷, Pro³⁸[Asp²⁸]exendin-4(1-39)-(Lys)₆-NH₂,
H-(Lys)₆-des Pro³⁶[Trp(O₂)²⁵, Asp²⁸]exendin-4(1-39)-Lys₆-NH₂,
H-des Asp²⁸Pro³⁶, Pro³⁷, Pro³⁸[Trp(O₂)²⁵]exendin-4(1-39)-NH₂,
H-(Lys)₆-des Pro³⁶, Pro³⁷, Pro” [Trp(O₂)²⁵, Asp²⁸]exendin-4(1-39)-NH₂,
H-Asn-(Glu)₅-des Pro³⁶, Pro³⁷, Pro³⁸[Trp(O₂)²⁵, Asp²⁸]exendin-4(1-39)-NH₂,
des Pro³⁶, Pro³⁷, Pro³⁸[Trp(O₂)²⁵, Asp²⁸]exendin-4(1-39)-(Lys)₆-NH₂,
H-(Lys)₆-des Pro³⁶, Pro³⁷, Pro³⁸[Trp(O₂)²⁵, Asp²⁸ ]exendin-4(1-39)-(Lys)₆-NH₂,
H-Asn-(Glu)₅-des Pro³⁶, Pro³⁷, Pro³⁸[Trp(O₂)²⁵, Asp²⁸]exendin-4(1-39)-(Lys)₆-NH₂,
H-(Lys)₆-des Pro³⁶[Met(O )¹⁴, Asp²⁸]exendin-4(1-39)-Lys₆-NH₂,
des Met(O)¹⁴Asp²⁸Pro ³⁶, Pro³⁷, Pro³⁸exendin-4(1-39)-NH₂,
H-(Lys)₆-des Pro³⁶, Pro ³⁷, Pro³⁸[Met(O)¹⁴, Asp²⁸]exendin-4(1-39)-NH₂,
H-Asn-(Glu)₅-des Pro³⁶, Pro³⁷, Pro³⁸[Met(O)¹⁴, Asp²⁸]exendin-4(1-39)-NH₂,
des Pro³⁶, Pro³⁷, Pro3⁸[Met(O)¹⁴, Asp²⁸]exendin-4(1-39)-(Lys)₆-NH₂,
H-(Lys)₆-des Pro³⁶, Pro³⁷, Pro³⁸[Met(O)¹⁴, Asp²⁸]exendin-4(1-39)-Lys₆-NH₂,
H-Asn-(Glu)₅des Pro³⁶, Pro³⁷, Pro³⁸[Met(O)¹⁴, Asp²⁸]exendin-4(1-39)-(Lys)₆-NH₂,
H-(Lys)₆-des Pro³⁶[Met(O)¹⁴, Trp(O₂)²⁵, Asp²⁸]exendin-4(1-39)-Lys₆-NH₂,
des Asp²⁸Pro³⁶, Pro³⁷, Pro³⁸[Met(O)¹⁴, Trp(O₂)²⁵]exendin-4(1-39)-NH₂,
H-(Lys)₆-des Pro³⁶, Pro³⁷, Pro³⁸[Met(O)¹⁴, Trp(O₂)²⁵, Asp²⁸]exendin-4(1-39)-NH₂,
H-Asn-(Glu)₅-des Pro³⁶, Pro³⁷, Pro³⁸[Met(O)¹⁴, Asp²⁸]exendin-4(1-39)-NH₂,
   des Pro³⁶, Pro³⁷, Pro³⁸[Met(O)¹⁴, Trp(O₂)²⁵, Asp²⁸]exendin-4(1-39)-(Lys)₆-N H₂,
H-(Lys)₆-des Pro³⁶, Pro³⁷, Pro³⁸[Met(O)¹⁴, Trp(O₂)²⁵, Asp²⁸]exendin-4(1-39)-(Lys)₆-NH₂,
H-Asn-(Glu)₅-des Pro³⁶, Pro³⁷, Pro³⁸[Met(O)¹⁴, Trp(O₂)²⁵, Asp²⁸]exendin-4(1-39)-(Lys)₆-NH₂,
   or a pharmacologically tolerable salt thereof.

The invention further provides a pharmaceutical formulation as described above, further comprising Arg³⁴, Lys²⁶(N^{ε}(γ-glutamyl(N^{α}-hexadecanoyl))) GLP-1 (7-37) [liraglutide] or a pharmacologically tolerable salt thereof.

The invention further provides a pharmaceutical formulation as described above, comprising the amino acid methionine, preferably in a concentration range of up to 10 mg/ml, particularly preferably of up to 3 mg/ml.

The invention further provides a process for preparing a formulation as described above, which comprises
(a) introducing the components into an aqueous solution and
(b) adjusting the pH.

The invention further provides for the use of a formulation as described above for treating diabetes mellitus.

The invention provides a medicament for treating diabetes mellitus, composed of a formulation as described above.

The preparation may further comprise preservatives (e.g., phenol, cresol, parabens), isotonicity agents (e.g., mannitol, sorbitol, lactose, dextrose, trehalose, sodium chloride, glycerol), buffer substances, salts, acids, alkalis and also further excipients. These substances may each be present individually or else as mixtures.

Glycerol, dextrose, lactose, sorbitol, and mannitol are typically present in the pharmaceutical preparation at a concentration of 100-250 mM, NaCl at a concentration of up to 150 mM. Buffer substances, such as phosphate buffer, acetate buffer, citrate, arginine, glycylglycine or TRIS (i.e., 2-amino-2-hydroxymethyl-1,3-propanediol) buffer, for example, and also corresponding salts, may be present at a concentration of 5-250 mM, preferably 10-100 mM. Further excipients may include salts or arginine.

The invention further provides a pharmaceutical formulation as described above, comprising the insulin analog at a concentration of 60-6000 nmol/ml (which corresponds approximately to a concentration of 0.35-70 mg/ml or 10-1000 units/ml), preferably at a concentration of 240-3000 nmol/ml (which corresponds approximately to a concentration of 1.4-35 mg/ml or 40-500 units/ml); and comprising the surfactant at a concentration of 5-200 μg/ml, preferably of 5-120 μg/ml, and more preferably of 20-75 μg/ml.

The invention further provides a pharmaceutical formulation as set out above, comprising glycerol and/or mannitol at a concentration of 100-250 mM, and/or NaCl preferably at a concentration of up to 150 mM.

The invention further provides a pharmaceutical formulation as set out above, comprising a buffer substance at a concentration of 5-250 mM.

The invention further provides a pharmaceutical insulin formulation which comprises further additions such as salts, for example, that retard the release of insulin. Mixtures of delayed-release insulins of this kind with formulations described above are also included in this.

The invention further provides a method for producing pharmaceutical formulations of this kind. Likewise provided by the invention, furthermore, is the use of such formulations for treating diabetes mellitus. The invention additionally provides for the use of or addition of surfactants as a stabilizer during the process of preparing insulin, insulin analogs or insulin derivatives or preparations thereof.

The specification is described below with reference to a number of examples, which are not intended to have any restrictive effect whatsoever.

### EXAMPLES

The examples below are intended to illustrate the concept of the invention, without having any restricting effect.

### LEGEND FOR THE FIGURES

FIG. 1: Blood sugar-lowering effect of new insulin analogs according to formula I in rats

FIG. 2: Blood sugar-lowering effect of new insulin analogs according to formula I in dogs

FIG. 3: Blood sugar-lowering effect of YKL205 in dogs

FIG. 4: Zinc dependence of the hypoglycemic effect of YKL205 in dogs

### EXAMPLE 1

### Studies on Evaluating the Optimum pH

The solution is prepared by introducing about 25% of injection-grade water. In succession, SAR161271 and the zinc chloride stock solution are added and stirred. Adding 1 M HCl at a pH of pH 2 dissolves SAR161271. The solution is stirred and then 1 M NaOH is added to adjust the pH to pH 3.75 (3.8). Injection-grade water is used to make up to 90% of the batch size. Added to this solution in succession with stirring are glycerol 85% and m-cresol. Injection-grade water is used to make up to the desired final weight. The solution is filtered using a filter attachment on a syringe. This mode of solution preparation was used to prepare formulations which were adjusted to the following pH levels: pH 3.0, 3.25, 3.5, 3.75, 4.0, and 4.5. A 3-month stability study was conducted using these formulations. Given below are the 2-month stability study results of the formulations with a pH of 3.0, 4.0, and 4.5.

| | | | | | | | t2 months; | | |
|---|---|---|---|---|---|---|---|---|---|
| | t0 | | | t2 months; 5° C. | | | 25° C./60% rH | | |
| | pH 3.5 | pH 4.0 | pH 4.5 | pH 3.5 | pH 4.0 | pH 4.5 | pH 3.5 | pH 4.0 | pH 4.5 |
| pH | 3.5 | 4.0 | 4.5 | 3.5 | 4.0 | 4.5 | 3.5 | 4.0 | 4.5 |
| Assay | 3.87 | 3.81 | 3.68 | 3.71 | 3.80 | 3.69 | 3.76 | 3.72 | 3.53 |
| SAR171271 | | | | | | | | | |
| [mg/ml] | | | | | | | | | |
| Other similar | 0.6/2.8 | 0.6/2.8 | 0.6/2.9 | 0.7/2.7 | 0.7/2.9 | 1.0/3.4 | 1.0/3.1 | 1.1/4.1 | 1.8/7.2 |
| impurities/ | | | | | | | | | |
| similar | | | | | | | | | |
| impurities | | | | | | | | | |
| total [%] | | | | | | | | | |
| Assay m-cresol | 2.8 | 2.5 | 2.9 | 2.7 | 2.5 | 2.8 | 2.7 | 2.4 | 2.8 |
| [mg/ml] | | | | | | | | | |
| HMWP [%] | 0.2 | 0.2 | 0.2 | 0.3 | 0.3 | 0.3 | 0.3 | 0.4 | 1.0 |
| Turbidity | clear | clear | clear | clear | clear | clear | clear | clear | clear |

The results show that, the more acidic the pH of the solution, the more stable it is.

### EXAMPLE 2

### Studies on the Choice of the Tonicity Agent

The solution was prepared as described in example 1. The results of the 2-month stability study with 2.5% glycerol vs. 0.8% NaCI are as follows:

### 2.5% Glycerol

Amount of SAR161271
2 M+5° C.: 3.56 mg/ml
2 M+25° C.: 3.46 mg/ml

Impurities
2 M+5° C.: 2.6%
2 M+25° C.: 3.8%

High Molecular Weight Proteins
2 M+5° C.: 0.2%
2 M+25° C.: 0.4%

### 0.8% NaCl

Amount of SARI 61271
2 M+5° C.: 3.52 mg/ml
2 M+25° C.: 3.49 mg/ml

Impurities

2 M+5° C.: 2.7%
2 M+25° C.: 4.8%

High Molecular Weight Proteins
2 M+5° C.: 0.2%
2 M+25° C.: 1.1%

Using the results from this study, glycerol was selected as the tonicity agent, at a concentration of 2.5%. The formulation is more stable as compared with 0.8% NaCl as tonicity agent. In addition, a precipitation, which was insoluble, was found when using NaCl during the preparation. For both substances the osmolarity was 290±30 mosmol/kg.

### EXAMPLE 3

### Studies on the Selection of the Preservative

The formulations described below were prepared as described in example 1. Formulations with different preservatives were passed to microbial quality control, where they underwent a preservative loading test (corresponding to Ph. Eur. 5.5 Criterion A and USP 29).
m-Cresol: 1.5, 1.8, 2.1, and 2.7 mg/ml
Phenol: 2.7 mg/ml
m-Cresol: 1.5 mg/ml and phenol: 0.6 mg/ml
Benzyl alcohol: 15 mg/ml

The preservative selected was m-cresol. The concentration of 2 mg/ml was selected, although just 1.5 mg/ml would have been sufficient for preservation. Nevertheless, the higher m-cresol concentration was selected on account of microbiological safety aspects and the specification laid down. In addition, the formulations (apart from 2.1 mg/ml of m-cresol) were designed for stability (3 months).

### EXAMPLE 4

### Formulation of Amidated Insulin Derivatives

Examples 4 to 8 serve only for the determination of the biological, pharmacological, and physicochemical properties of insulin analogs of formula I, involving first the provision of formulations thereof (example 4) and then the conduct of corresponding tests (examples 5 to 8). A solution with the compounds was prepared as follows: the insulin analog of the invention was dissolved with a target concentration of 240±5 μM in 1 mM hydrochloric acid with 80 μg/ml zinc (as zinc chloride).

The compositions used as dissolution medium were as follows:
a) 1 mM hydrochloric acid
b) 1 mM hydrochloric acid, 5 μg/ml zinc (added as zinc chloride or hydrochloric acid)
c) 1 mM hydrochloric acid, 10 μg/ml zinc (added as zinc chloride or hydrochloric acid)
d) 1 mM hydrochloric acid, 15 μg/ml zinc (added as zinc chloride or hydrochloric acid)
e) 1 mM hydrochloric acid, 30 μg/ml zinc (added as zinc chloride or hydrochloric acid)
f) 1 mM hydrochloric acid, 80 μg/ml zinc (added as zinc chloride or hydrochloric acid)
g) 1 mM hydrochloric acid, 120 μg/ml zinc (added as zinc chloride or hydrochloric acid)

For this purpose, an amount of the freeze-dried material higher by around 30% than the amount needed on the basis of the molecular weight and the target concentration was first weighed out. Thereafter the existing concentration was determined by means of analytical HPLC and the solution was then made up with 5 mM hydrochloric acid with 80 μg/ml zinc to the volume needed in order to achieve the target concentration. If necessary, the pH was readjusted to 3.5±0.1. Following final analysis by HPLC to ensure the target concentration of 240±5 μM, the completed solution was transferred, using a syringe having a 0.2 μm filter attachment, into a sterile vial which was closed with a septum and a crimped cap. For the short-term, single testing of the insulin derivatives of the invention, there was no optimization of the formulations, in relation, for example, to addition of isotonic agents, preservatives or buffer substances.

### EXAMPLE 5

### Evaluation of the Blood Sugar-Reducing Action of New Insulin Analogs in Rats

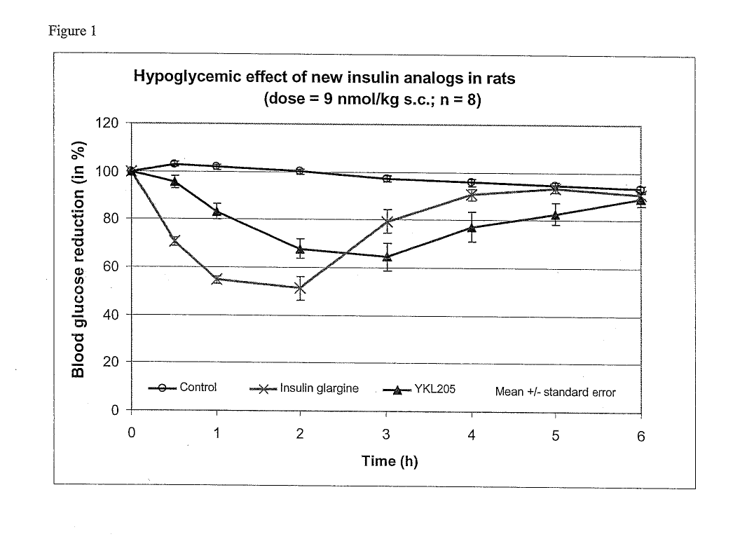

The blood sugar-lowering effect of selected new insulin analogs is tested in healthy male normoglycemic Wistar rats. Male rats receive a subcutaneous injection of a dose of 9 nmol/kg of an insulin analog. Immediately before the injection of the insulin analog and at regular intervals for up to eight hours after injection, blood samples are taken from the animals, and their blood sugar content determined. The experiment shows clearly (cf. FIG. 1) that the insulin analog of the invention leads to a significantly retarded onset of action and to a longer, uniform duration of action.

### EXAMPLE 6

### Evaluation of the Blood Sugar-Reducing Action of New Insulin Analogs in Dogs

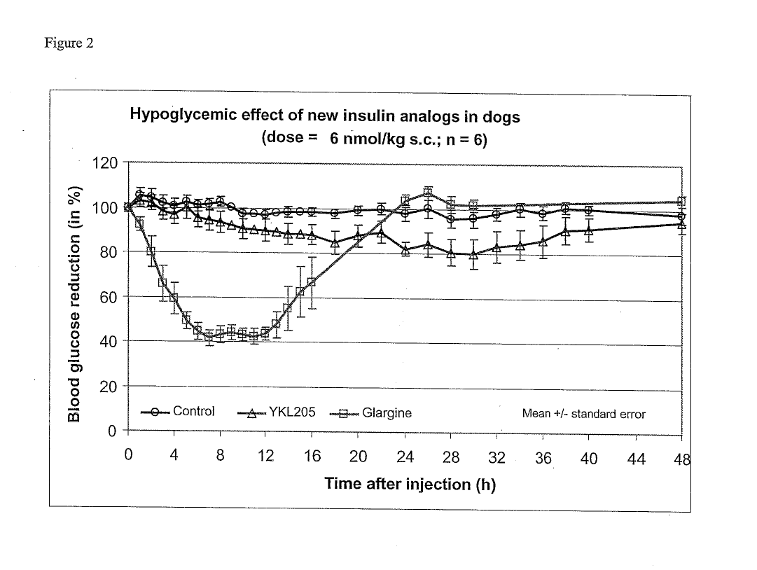

The blood sugar-lowering effect of selected new insulin analogs is tested in healthy male normoglycemic beagles. Male animals receive a subcutaneous injection of a dose of 6 nmol/kg of an insulin analog. Immediately before the injection of the insulin analog and at regular intervals for up to forty-eight hours after injection, blood samples are taken from the animals, and their blood sugar content determined. The experiment shows clearly (cf. FIG. 2) that the insulin analog of the invention that is used leads to a significantly retarded onset of action and to a longer, uniform duration of action.

### EXAMPLE 7

### Evaluation of the Blood Sugar-Reducing Action in Dogs with Twofold-Increased Dose

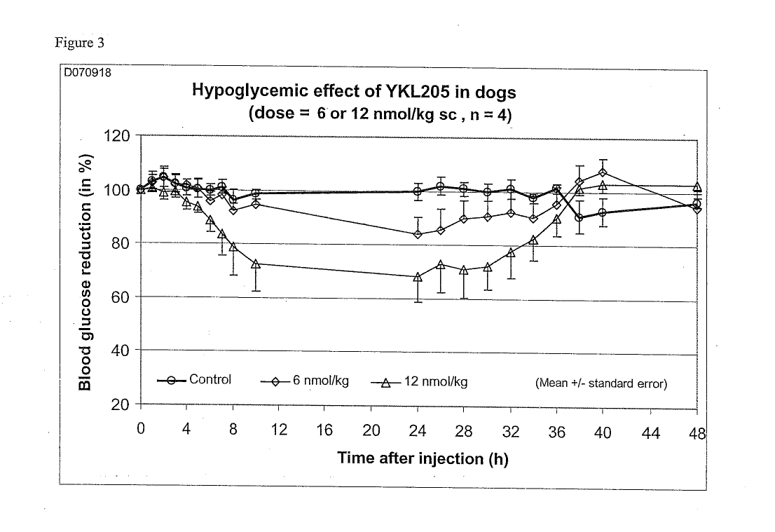

The blood sugar-lowering effect of selected new insulin analogs is tested in healthy male normoglycemic beagles. Male animals receive a subcutaneous injection of a dose of 6 nmol/kg and 12 nmol/kg of an insulin analog. Immediately before the injection of the insulin analog and at regular intervals for up to forty-eight hours after injection, blood samples are taken from the animals, and their blood sugar content determined. The experiment shows clearly (cf. FIG. 3) that the insulin analog of the invention that is used has a dose-dependent effect, but that, despite the twofold-increased dose, the effect profile is flat, i.e., there is no pronounced low point (nadir) observed. From this it may be inferred that the insulins of the invention, in comparison to known retarded insulins, lead to significantly fewer hypoglycemic events.

### EXAMPLE 8

### Evaluation of the Blood Sugar-Reducing Effect in Dogs with Different Concentrations of Zinc in the Formulation

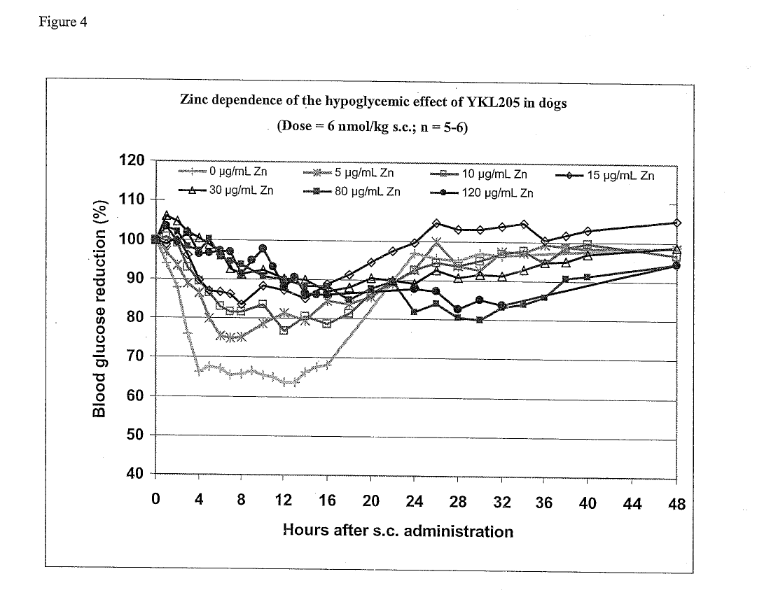

The experiments were carried out as described in example 35. FIG. 4 shows the result. Accordingly, the time/activity curve of the insulin analog of the invention can be influenced through the amount of zinc ions in the formulation, with the same concentration of insulin, in such a way that a rapid onset of action is observed at zero or low zinc content and the action persists over 24 hours, whereas, with a higher zinc content, a flat onset of action is observed and the insulin effect persists for much longer than 24 hours.

### EXAMPLE 9

### Formulation of Amidated Insulin Derivatives

Examples 9 to 11 serve only for the determination of the biological, pharmacological, and physicochemical properties of insulin analogs of formula II, involving first the provision of formulations thereof (example 9) and then the conduct of corresponding tests (examples 10 and 11). The insulin analog of the invention was dissolved with a target concentration of 240±5 μM in 1 mM hydrochloric acid with 80 μg/ml zinc (as zinc chloride). For this purpose, an amount of the freeze-dried material higher by around 30% than the amount needed on the basis of the molecular weight and the target concentration was first weighed out. Thereafter the existing concentration was determined by means of analytical HPLC and the solution was then made up with 5 mM hydrochloric acid with 80 μg/ml zinc to the volume needed in order to achieve the target concentration. If necessary, the pH was readjusted to 3.5±0.1. Following final analysis by HPLC to ensure the target concentration of 240±5 μM, the completed solution was transferred, using a syringe having a 0.2 μm filter attachment, into a sterile vial which was closed with a septum and a crimped cap. For the short-term, single testing of the insulin derivatives of the invention, there was no optimization of the formulations, in relation, for example, to addition of isotonic agents, preservatives or buffer substances.

### EXAMPLE 10

### Evaluation of the Blood Sugar-Reducing Action of New Insulin Analogs in Rats

The blood sugar-lowering effect of selected new insulin analogs is tested in healthy male normoglycemic Wistar rats. Male rats receive a subcutaneous injection of a dose of 9 nmol/kg of an insulin analog. Immediately before the injection of the insulin analog and at regular intervals for up to eight hours after injection, blood samples are taken from the animals, and their blood sugar content determined. The experiment shows clearly (cf. FIG. 4) that the insulin analog of the invention leads to a significantly retarded onset of action and to a longer, uniform duration of action.

### EXAMPLE 11

### Evaluation of the Blood Sugar-Reducing Action of New Insulin Analogs in Dogs

The blood sugar-lowering effect of selected new insulin analogs is tested in healthy male normoglycemic beagles. Male animals receive a subcutaneous injection of a dose of 6 nmol/kg of an insulin analog. Immediately before the injection of the insulin analog and at regular intervals for up to forty-eight hours after injection, blood samples are taken from the animals, and their blood sugar content determined. The experiment shows clearly that the insulin analog of the invention leads to a significantly retarded, flat onset of action and to a longer, uniform duration of action.

1. Aqueous An aqueous pharmaceutical formulations formulation comprising an insulin analog of the formula I 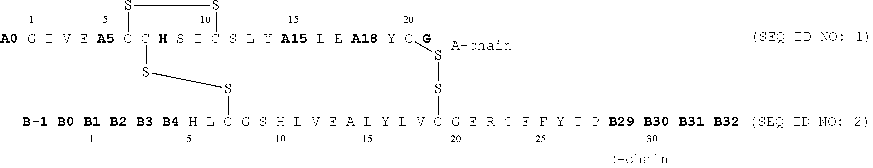
where
A0 is Lys or Arg;
A5 is Asp, Gln or Glu;
A15 is Asp, Glu or Gln;
A 18 is Asp, Glu or Asn;
B-1 is Asp, Glu or an amino group;
B0 is Asp, Glu or a chemical bond;
B1 is Asp, Glu or Phe;
B2 is Asp, Glu or Val;
B3 is Asp, Glu or Asn;
B4 is Asp, Glu or Gln;
B29 is Lys or a chemical bond;
B30 is Thr or a chemical bond;
B31 is Arg, Lys or a chemical bond;
B32 is Arg-amide, Lys-amide or an amino group,
where two amino acid residues of the group containing A5, A15, A18, B-1, B0, B1, B2, B3, and B4, simultaneously and independently of one another, are Asp or Glu, or a pharmacologically tolerable salt thereof; and comprising
0.001 to 0.2 mg/ml of zinc,
0.1 to 5.0 mg/ml of a preservative, and
5.0 to 100 mg/ml of an isotonicity agent, and
whose pH is 5 or less.

2. The pharmaceutical formulation as claimed in claim 1, in which the insulin analog is selected from the group containing:
Arg (A0), His (A8), Glu (A5), Asp (A18), Gly (A21), Arg (B31), Arg (B32)-NH₂human insulin,
Arg (A0), His (A8), Glu (A5), Asp (A18), Gly (A21), Arg (B31), Lys (B32)-NH₂human insulin,
Arg (A0), His (A8), Glu (A15), Asp (A18), Gly (A21), Arg (B31), Arg (B32)-NH₂human insulin,
Arg (A0), His (A8), Glu (A15), Asp (A18), Gly (A21), Arg (B31), Lys (B32)-NH₂human insulin,
Arg (A0), His (A8), Glu(A5), Glu (A15), Gly (A21), Arg (B31), Arg (B32)-NH₂human insulin,
Arg (A0), His (A8), Glu (A5), Glu (A15), Gly (A21), Arg (B31), Lys (B32)-NH₂ human insulin,
Arg (A0), His(A8), Glu (A5), Gly (A21), Asp (B3), Arg (B31), Arg (B32)-NH₂human insulin,
Arg (A0), His(A8), Glu (A5), Gly (A21), Asp (B3), Arg (B31), Lys (B32)-NH₂human insulin,
Arg (A0), His (A8), Glu (A15), Gly (A21), Asp (B3), Arg (B31), Arg (B32)-NH₂ human insulin,
Arg (A0), His (A8), Glu (A15), Gly (A21), Asp (B3), Arg (B31), Lys (B32)-NH₂human insulin,
Arg (A0), His (A8), Asp (A18), Gly (A21), Asp (B3), Arg (B31), Arg (B32)—NH₂human insulin,
Arg (A0), His (A8), Asp (A18), Gly (A21), Asp (B3), Arg (B31), Lys (B32)-NH₂human insulin,
Arg (A0), His(A8), Gly (A21), Asp (B3), Glu (B4), Arg (B31), Arg (B32)-NH₂human insulin,
Arg (A0), His (A8), Gly (A21), Asp (B3), Glu (B4), Arg (B31), Lys (B32)-NH₂ human insulin,
Arg (A0), His (A8), Glu (A5), Gly (A21), Glu (B4), Arg (B31), Arg (B32)-NH₂human insulin,
Arg (A0), His (A8), Glu (A5), Gly (A21), Glu (B4), Arg (B31), Lys (B32)-NH₂ human insulin,
Arg (A0), His (A8), Glu (A15), Gly (A21), Glu (B4), Arg (B31), Arg (B32)-NH₂human insulin,
Arg (A0), His (A8), Glu (A15), Gly (A21), Glu (B4), Arg (B31), Lys (B32)-NH₂human insulin,
Arg (A0), His (A8), Asp (A18), Gly (A21), Glu (B4), Arg (B31), Arg (B32)-NH₂human insulin,
Arg (A0), His (A8), Asp (A18), Gly (A21), Glu (B4), Arg (B31), Lys (B32)-NH₂ human insulin,
Arg (A0), His (A8), Glu (A5), Gly (A21), Glu (B0), Arg (B31), Arg (B32)-NH₂human insulin,
Arg (A0), His (A8), Glu (A5), Gly (A21), Glu (B0), Arg (B31), Lys (B32)-NH₂ human insulin,
Arg (A0), His (A8), Glu (A15), Gly (A21), Glu (B0), Arg (B31), Arg (B32)-NH₂human insulin,
Arg (A0), His (A8), Glu (A15), Gly (A21), Glu (B0), Arg (B31), Lys (B32)-NH₂human insulin,
Arg (A0), His (A8), Asp (A18), Gly (A21), Glu (B0), Arg (B31), Arg (B32)-NH₂human insulin,
Arg (A0), His (A8), Asp (A18), Gly (A21), Glu (B0), Arg (B31), Lys (B32)-NH₂human insulin,
Arg (A0), His (A8), Glu (A5), Gly (A21), Asp (B1), Arg (B31), Arg (B32)-NH₂human insulin,
Arg (A0), His (A8), Glu (A5), Gly (A21), Asp (B1), Arg (B31), Lys (B32)-NH₂human insulin,
Arg (A0), His (A8), Glu (A15), Gly (A21), Asp (B1), Arg (B31), Arg(B32)-NH₂human insulin,
Arg (A0), His (A8), Glu (A15), Gly (A21), Asp (B1), Arg (B31), Lys (B32)-NH₂human insulin,
Arg (A0), His (A8), Asp (A18), Gly (A21), Asp (B1), Arg (B31), Arg (B32)-NH₂human insulin,
Arg (A0), His (A8), Asp (A18), Gly (A21), Asp (B1), Arg (B31), Lys (B32)-NH₂human insulin,
Arg (A0), His (A8), Gly (A21), Glu (B0), Asp (B1), Arg (B31), Arg (B32)-NH₂human insulin,
Arg (A0), His (A8), Gly (A21), Glu (B0), Asp (B1), Arg (B31), Lys (B32)-NH₂human insulin,
Arg (A0), His (A8), Asp (A18), Gly (A21), Asp (B3), Arg (B30), Arg (B31)-NH₂human insulin,
Arg (A0), His (A8), Asp (A18), Gly (A21), Asp (B3), Arg (B30), Lys (B31)-NH₂ human insulin.

3. The pharmaceutical formulation as claimed in claim 1, wherein the preservative is selected from a group containing phenol, m-cresol, chlorocresol, benzyl alcohol, and parabens.

4. The pharmaceutical formulation as claimed in claim 1, wherein the isotonicity agent is selected from a group containing mannitol, sorbitol, lactose, dextrose, trehalose, sodium chloride, and glycerol.

5. The pharmaceutical formulation as claimed in claim 1, wherein said formulation has a pH in the range of pH 2.5-4.5.

6. The pharmaceutical formulation as claimed in claim 1, wherein said formulation has having a pH in the range of pH 3.0-4.0.

7. The pharmaceutical formulation as claimed in claim 1, wherein said formulation has a pH in the region of pH 3.75.

8. The pharmaceutical formulation as claimed in wherein said insulin, insulin analog and/or insulin derivative are present in a concentration of 240-3000 nmol/ml.

9. The pharmaceutical formulation as claimed in claim 1, comprising glycerol at a concentration of 20 to 30 mg/ml.

10. The pharmaceutical formulation as claimed in claim 1, comprising glycerol at a concentration of 25 mg/ml.

11. The pharmaceutical formulation as claimed in claim 1, comprising m-cresol at a concentration of 1 to 3 mg/ml.

12. The pharmaceutical formulation as claimed in claim 1, comprising m-cresol at a concentration of 2 mg/ml.

13. The pharmaceutical formulation as claimed in claim 1, comprising zinc at a concentration of 0.01 or 0.03 or 0.08 mg/ml.

14. The pharmaceutical formulation as claimed in claim 1, further comprising a glucagon-like peptide-1 (GLP1) or an analog or derivative thereof, or exendin-3 and/or -4 or an analog or derivative thereof.

15. The pharmaceutical formulation as claimed in claim 14, further comprising exendin-4.

16. The pharmaceutical formulation as claimed in claim 14, wherein said analog of exendin-4 is selected from a group containing
H-desPro³⁶-exendin-4-Lys₆-NH₂,
H-des(Pro^{36,37})-exendin-4-Lys₄-NH₂ and
H-des(Pro^{36,37})-exendin-4-Lys₅-NH₂,
or a pharmacologically tolerable salt thereof

17. The pharmaceutical formulation as claimed in claim 14, wherein said analog of exendin-4 is selected from a group containing
desPro³⁶[Asp²⁸]exendin-4 (1-39),
desPro³⁶[IsoAsp²⁸]exendin-4 (1-39),
desPro³⁶[Met(O)¹⁴, Asp²⁸]exendin-4 (1-39),
desPro³⁶[Met(O)¹⁴, IsoAsp²⁸]exendin-4 (1-39),
desPro³⁶[Trp(O₂)²⁵, Asp²⁸]exendin-2 (1-39),
desPro³⁶[Trp(O₂)²⁵, IsoAsp²⁸]exendin-2 (1-39),
desPro³⁶[Met(O)¹⁴Trp(O₂)²⁵, Asp²⁸]exendin-4 (1-39) and
desPro³⁶[Met(O)¹⁴Trp(O₂)²⁵, IsoAsp²⁸]exendin-4 (1-39),
or a pharmacologically tolerable salt thereof

18. The pharmaceutical formulation as claimed in claim 17, wherein peptide Lys₆-NH₂ is attached to the C-termini of the analogs of exendin-4.

19. The pharmaceutical formulation as claimed in claim 14, wherein said analog of exendin-4 is selected from the group containing
H-(Lys)₆-des Pro³⁶[Asp²⁸]exendin-4(1-39)-Lys₆-NH₂
des Asp²⁸Pro³⁶, Pro³⁷, Pro³⁸exendin-4(1-39)-NH₂,
H-(Lys)₆-des Pro³⁶, Pro³⁷, Pro³⁸[Asp²⁸]exendin-4(1-39)-NH₂,
H-Asn-(Glu)₅des Pro³⁶, Pro³⁷, Pro³⁸[Asp²⁸]exendin-4(1-39)-NH₂,
des Pro³⁶, Pro³⁷, Pro³⁸[Asp²⁸]exendin-4(1-39)-(Lys)₆-NH₂,
H-(Lys)₆-des Pro³⁶, Pro³⁷, Pro³⁸[Asp²⁸]exendin-4(1-39)-(Lys)₆-NH₂,
H-Asn-(Glu)₅-des Pro³⁶, Pro³⁷, Pro³⁸[Asp²⁸]exendin-4(1-39)-(Lys)₆-NH₂,
H-(Lys)₆-des Pro³⁶[Trp(O₂)²⁵, Asp²⁸[exendin-4(1-39)-Lys₆-NH₂,
H-des Asp²⁸Pro³⁶, Pro³⁷, Pro³⁸[Trp(O₂)²⁵]exendin-4(1-39)-NH₂,
H-(Lys)₆-des Pro³⁶, Pro³⁷, Pro³⁸[Trp(O₂)²⁵, Asp²⁸]exendin-4(1-39)-NH₂,
H-Asn-(Glu)₅-des Pro³⁶, Pro³⁷, Pro³⁸[Trp(O₂)²⁵, Asp²⁸]exendin-4(1-39)-NH₂,
des Pro³⁶, Pro³⁷, Pro³⁸[Trp(O₂)²⁵, Asp²⁸]exendin-4(1-39)-(Lys)₆-NH₂,
H-(Lys)₆-des Pro³⁶, Pro³⁷, Pro³⁸[Trp(O₂)²⁵, Asp²⁸]exendin-4(1-39)-(Lys)₆-NH₂,
H-Asn-(Glu)₅-des Pro³⁶, Pro³⁷, Pro³⁸[Trp(O₂)²⁵, Asp²⁸]exendin-4(1-39)-(Lys)₆-NH₂,
H-(Lys)₆-des Pro³⁶[Met(O)¹⁴, Asp²⁸]exendin-4(1-39)-Lys₆-NH₂,
des Met(O)¹⁴Asp²⁸Pro ³⁶, Pro³⁷, Pro³⁸exendin-4(1-39)-NH₂,
H-(Lys)₆-des Pro³⁶, Pro³⁷, Pro³⁸[Met(O)¹⁴, Asp²⁸]exendin-4(1-39)-NH₂,
H-Asn-(Glu)₅-des Pro³⁶, Pro³⁷, Pro³⁸[Met(O)^{14, Asp}²⁸]exendin-4(1-39)-NH₂,
des Pro³⁶, Pro³⁷, Pro³⁸[Met(O)¹⁴, Asp²⁸]exendin-4(1-39)-(Lys)₆-NH₂,
H-(Lys)₆-des Pro³⁶, Pro³⁷, Pro³⁸[Met(O)¹⁴, Asp²⁸]exendin-4(1-39)-Lys₆-NH₂,
H-Asn-(Glu)₅des Pro³⁶, Pro³⁷, Pro³⁸[Met(O)¹⁴, Asp²⁸]exendin-4(1-39)-(Lys)₆-NH₂,
H-(Lys)₆-des Pro³⁶[Met(O)¹⁴, Trp(O₂)²⁵, Asp²⁸]exendin-4(1-39)-Lys₆-NH₂,
des Asp²⁸Pro³⁶, Pro³⁷, Pro³⁸[Met(O)¹⁴, Trp(O₂)²⁵]exendin-4(1-39)-NH₂,
H-(Lys)₆-des Pro³⁶, Pro³⁷, Pro³⁸[Met(O)¹⁴, Trp(O₂)²⁵, Asp²⁸[exendin-4(1-39)-NH₂,
H-Asn-(Glu)₅-des Pro³⁶, Pro³⁷, Pro³⁸[Met(O)¹⁴, Asp²⁸]exendin-4(1-39)-NH₂,
des Pro³⁶, Pro³⁷, Pro³⁸[Met(O)¹⁴, Trp(O₂)²⁵, Asp²⁸]exendin-4(1-39)-(Lys)₆-NH₂,
H-(Lys)₆-des Pro³⁶, Pro³⁷, Pro³⁸[Met(O)¹⁴, Trp(O₂)²⁵, Asp²⁸]exendin-4(1-39)-(Lys)₆-NH₂,
H-Asn-(Glu)₅-des Pro³⁶, Pro³⁷, Pro³⁸[Met(O)¹⁴, Trp(O₂)²⁵, Asp²⁸]exendin-4(1-39)-(Lys)₆-NH₂,
or a pharmacologically tolerable salt thereof

20. The pharmaceutical formulation as claimed in claim 14, further comprising Arg³⁴, Lys²⁶(N^{ε}(γ-glutamyl(N^{α}-hexadecanoyl))) GLP-1 (7-37) [liraglutide] or a pharmacologically tolerable salt thereof.

21. The pharmaceutical formulation as claimed in claim 1, comprising methionine.

22. The pharmaceutical formulation as claimed in claim 21, comprising methionine in the concentration range of up to 10 mg/ml.

23. The pharmaceutical formulation as claimed in claim 22, comprising methionine in the concentration range of up to 3 mg/ml.

24. A process for preparing the pharmaceutical formulation of claim 1, comprising
(a) introducing the components into an aqueous solution and
(b) adjusting the pH.

25. A method of treating diabetes mellitus in a patient in need thereof comprising administering to said patient a therapeutically effective amount of the pharmaceutical formulation of claim 1.

26. (canceled)
